(19) United States
(12) Patent Application Publication
(10) Pub. No.: US 2011/0160586 A1
(43) Pub. Date: Jun. 30, 2011

(54) SYSTEMS AND METHODS FOR MULTI-FREQUENCY IMAGING OF PATIENT TISSUE USING INTRAVASCULAR ULTRASOUND IMAGING SYSTEMS

(75) Inventors: Wenguang Li, Campbell, CA (US); Tat-Jin Teo, Sunnyvale, CA (US); Shashidhar Sathyanarayana, Pleasanton, CA (US)

(73) Assignee: Boston Scientific SciMed, Inc., Maple Grove, MN (US)

(21) Appl. No.: 12/979,660

(22) Filed: Dec. 28, 2010

Related U.S. Application Data
(60) Provisional application No. 61/290,842, filed on Dec. 29, 2009.

(51) Int. Cl. A61B 8/14

(52) U.S. Cl. 600/443

(57) ABSTRACT

A method for imaging patient tissue using an intravascular ultrasound image includes inserting a catheter into a patient blood vessel. The catheter includes at least one transducer configured and arranged for insertion into a lumen of the catheter. Acoustic signals are transmitted from the at least one transducer along a series of scan lines towards patient tissue between incremental rotations of the at least one transducer. The transmitted acoustic signals include first acoustic signals having first frequency bandwidths centered at a first center frequency and second acoustic signals having second frequency bandwidths centered at a second center frequency. Corresponding echo signals reflected from patient tissue are received, transformed, processed, and displayed.

### CROSS-REFERENCE TO RELATED APPLICATIONS

This application claims the benefit under 35 U.S.C. §119(e) of U.S. Provisional Patent Application Ser. No. 61/290,842 filed on Dec. 29, 2009, which is incorporated herein by reference.

### TECHNICAL FIELD

The present invention is directed to the area of intravascular ultrasound imaging systems and methods of making and using the systems. The present invention is also directed to systems and methods for imaging patient tissue with intravascular ultrasound imaging systems by transmitting acoustic signals at multiple frequencies, as well as methods of making and using the intravascular ultrasound imaging systems.

### BACKGROUND

Intravascular ultrasound (“IVUS”) imaging systems have proven diagnostic capabilities for a variety of diseases and disorders. For example, IVUS imaging systems have been used as an imaging modality for diagnosing blocked blood vessels and providing information to aid medical practitioners in selecting and placing stents and other devices to restore or increase blood flow. IVUS imaging systems have been used to diagnose atheromatous plaque build-up at particular locations within blood vessels. IVUS imaging systems can be used to determine the existence of an intravascular obstruction or stenosis, as well as the nature and degree of the obstruction or stenosis. IVUS imaging systems can be used to visualize segments of a vascular system that may be difficult to visualize using other intravascular imaging techniques, such as angiography, due to, for example, movement (e.g., a beating heart) or obstruction by one or more structures (e.g., one or more blood vessels not desired to be imaged). IVUS imaging systems can be used to monitor or assess ongoing intravascular treatments, such as balloon angioplasty and stent placement in real (or almost real) time. Moreover, IVUS imaging systems can be used to monitor one or more heart chambers.

IVUS imaging systems have been developed to provide a diagnostic tool for visualizing a variety is diseases or disorders. An IVUS imaging system can include a control module (with a pulse generator, an image processor, and a monitor), a catheter, and one or more transducers disposed in the catheter. The transducer-containing catheter can be positioned in a lumen or cavity within, or in proximity to, a region to be imaged, such as a blood vessel wall or patient tissue in proximity to a blood vessel wall. The pulse generator in the control module generates electrical signals that are delivered to the one or more transducers and transformed to acoustic signals that are transmitted through patient tissue. Reflected signals of the transmitted acoustic signals are absorbed by the one or more transducers and transformed to electric signals. The transformed electric signals are delivered to the image processor and converted to an image displayable on the monitor.

### BRIEF SUMMARY

In one embodiment, a method for imaging patient tissue using an intravascular ultrasound image includes inserting a catheter into a patient blood vessel. The catheter includes an imaging core configured and arranged for insertion into a lumen of the catheter and disposition at a distal end of the catheter. The imaging core includes at least one ultrasound transducer configured and arranged for transforming applied electrical signals to a plurality of acoustic signals. The acoustic signals are transmitted along a series of scan lines towards patient tissue between incremental rotations of the at least one transducer. A plurality of the acoustic signals transmitted along the series of scan lines are first acoustic signals having first frequency bandwidths centered at a first center frequency. A plurality of the acoustic signals transmitted along the series of scan lines are second acoustic signals having second frequency bandwidths centered at a second center frequency that is lower than the first center frequency. Corresponding echo signals reflected from patient tissue are received for each scan line. The received echo signals are transformed to electrical signals. The received electrical signals are processed from the at least one transducer to form at least one image. The at least one image is displayed on a display.

In another embodiment, a computer-readable medium includes processor-executable instructions for generating an intravascular ultrasound image formed in response to transmission of a plurality of acoustic signals from a transducer. The processor-executable instructions when installed onto a device enable the device to perform actions, including transmitting the acoustic signals along a series of scan lines towards patient tissue between incremental rotations of the at least one transducer. At least some of the acoustic signals transmitted along the series of scan lines are first frequency acoustic signals having first frequency bandwidths centered at a first center frequency. At least some of the acoustic signals transmitted along the series of scan lines are second frequency acoustic signals having second frequency bandwidths centered at a second center frequency that is lower than the first center frequency. Corresponding echo signals reflected from patient tissue are received for each scan line. The received echo signals are transformed to electrical signals. The received electrical signals are processed from the at least one transducer to form at least one image. The at least one image is displayed on a display.

In yet another embodiment, a catheter-based intravascular ultrasound imaging system includes at least one imager disposed in a catheter at least partially insertable into a patient blood vessel. The at least one imager is coupled to a control module. A processor is in communication with the control module. The processor executes processor-readable instructions that enable actions, including transmitting acoustic signals along a series of scan lines towards patient tissue between incremental rotations of the at least one imager. At least some of the acoustic signals transmitted along the series of scan lines are first frequency acoustic signals having first frequency bandwidths centered at a first center frequency. At least some of the acoustic signals transmitted along the series of scan lines are second frequency acoustic signals having second frequency bandwidths centered at a second center frequency that is lower than the first center frequency. Corresponding echo signals reflected from patient tissue are received for each scan line. The received echo signals are transformed to electrical signals. The received electrical signals are processed from the at least one transducer to form at least one image. The at least one image is displayed on a display.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an intravascular ultrasound imaging system, according to the invention;

FIG. 2 is a schematic side view of one embodiment of a catheter of an intravascular ultrasound imaging system, according to the invention;

FIG. 3 is a schematic perspective view of one embodiment of a distal end of the catheter shown in FIG. 2 with an imaging core disposed in a lumen defined in the catheter, according to the invention;

FIG. 4 is a schematic longitudinal cross-sectional view of a portion of a blood vessel with an exemplary atheroma;

FIG. 5 is a schematic longitudinal cross-sectional view of the portion of the blood vessel shown in FIG. 4 with an atheroma with a ruptured fibrous cap;

FIG. 6A is a schematic longitudinal cross-sectional view of the portion of the blood vessel shown in FIG. 4 with an occluding thrombus formed in a fibrous cap rupture;

FIG. 6B is a schematic longitudinal cross-sectional view of the portion of the blood vessel shown in FIG. 4 with a detached thrombus;

FIG. 7 is a schematic transverse cross-sectional view of another embodiment of an atheroma disposed in a blood vessel;

FIG. 8 is a schematic view of one embodiment of an IVUS image of an atheroma disposed in a blood vessel, the IVUS image generated from acoustic signals having a high-frequency, according to the invention;

FIG. 9A is a graph showing spectra of multiple acoustic signals output during an imaging procedure, each acoustic signal having a different center frequency and bandwidth, according to the invention;

FIG. 9B is a graph showing spectra of echo signals received after reflection of some of the acoustic signals of FIG. 9A from patient tissue, according to the invention;

FIG. 10A is a schematic view of one embodiment of a first IVUS image showing an atheroma within a blood vessel, the first IVUS image obtained using acoustic signals transmitted at a low frequency, according to the invention;

FIG. 10B is a schematic view of one embodiment of a second IVUS image showing the atheroma of FIG. 10A within the blood vessel of FIG. 10A, the second IVUS image obtained using acoustic signals transmitted at a high frequency that is greater than the low frequency of FIG. 10A, according to the invention;

FIG. 11A is a graph showing spectra of echo signals received after reflection of acoustic signals from patient tissue, the acoustic signals transmitted at a plurality of different frequencies, according to the invention;

FIG. 11B is a schematic view of one embodiment of a first IVUS image showing an atheroma within a blood vessel, the first IVUS image obtained using acoustic signals transmitted at a single wideband frequency, according to the invention;

FIG. 11C is a schematic view of one embodiment of a second IVUS image showing the atheroma of FIG. 11B within the blood vessel of FIG. 11B, the second IVUS image obtained using acoustic signals transmitted at a high frequency and acoustic signals transmitted at a low frequency, according to the invention; and

FIG. 12 is a flow diagram showing one exemplary embodiment of an enhanced IVUS imaging procedure for penetrating a necrotic region of an atheroma during an intravascular imaging procedure, according to the invention.

### DETAILED DESCRIPTION

The present invention is directed to the area of intravascular ultrasound imaging systems and methods of making and using the systems. The present invention is also directed to systems and methods for imaging patient tissue with intravascular ultrasound imaging systems by transmitting acoustic signals at multiple frequencies, as well as methods of making and using the intravascular ultrasound imaging systems.

The methods, systems, and devices described herein may be embodied in many different forms and should not be construed as limited to the embodiments set forth herein. Accordingly, the methods, systems, and devices, or portions thereof, described herein may take the form of an entirely hardware embodiment, an entirely software embodiment or an embodiment combining software and hardware aspects. Many of the steps of the methods described herein can be performed using any type of computing device, such as a computer, that includes a processor or any combination of computing devices where each device performs at least part of the process.

Suitable computing devices typically include mass memory and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

Methods of communication between devices or components of a system can include both wired and wireless (e.g., RF, optical, or infrared) communications methods and such methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

Suitable intravascular ultrasound (“IVUS”) imaging systems include, but are not limited to, one or more transducers disposed on a distal end of a catheter configured and arranged for percutaneous insertion into a patient. Examples of IVUS imaging systems with catheters are found in, for example, U.S. Pat. Nos. 7,306,561; and 6,945,938; as well as U.S. Patent Application Publication Nos. 20060253028; 20070016054; 20070038111; 20060173350; and 20060100522, all of which are incorporated by reference.

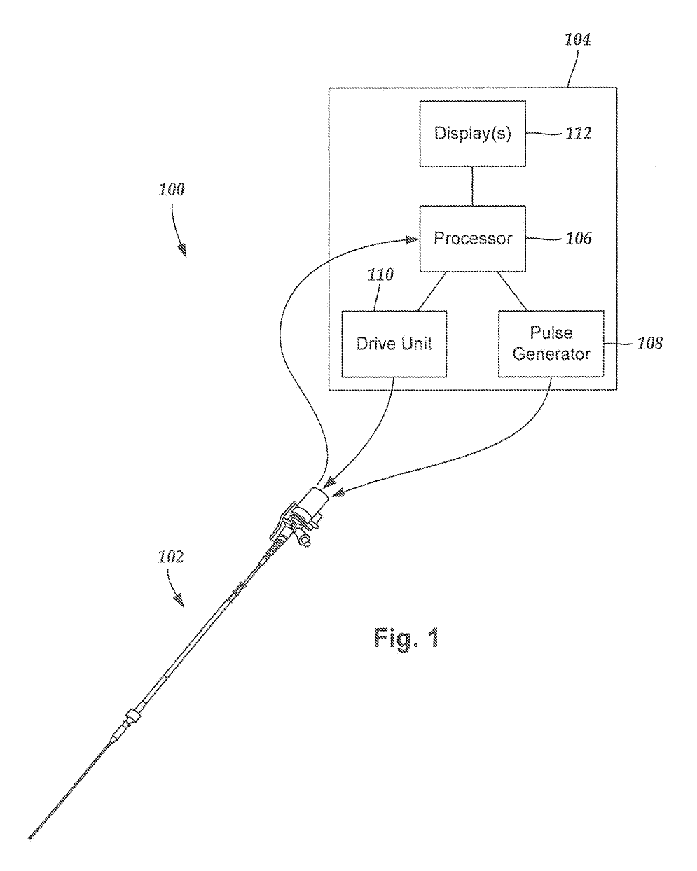

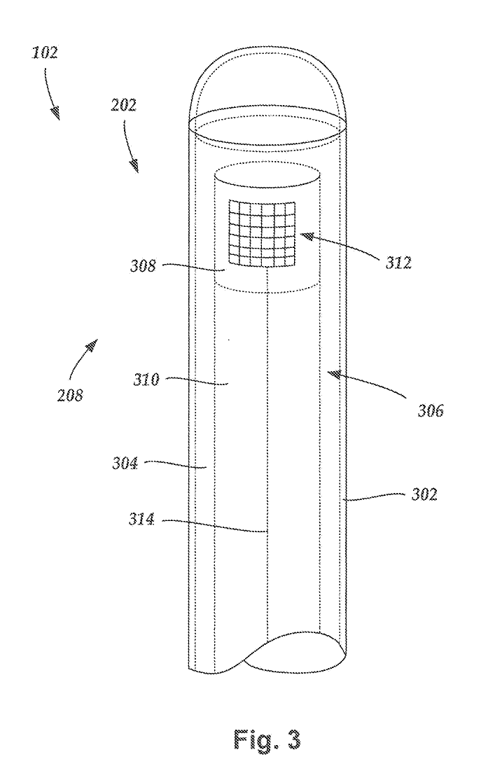

FIG. 1 illustrates schematically one embodiment of an IVUS imaging system 100. The IVUS imaging system 100 includes a catheter 102 that is coupleable to a control module 104. The control module 104 may include, for example, a processor 106, a pulse generator 108, a drive unit 110, and one or more displays 112. In at least some embodiments, the pulse generator 108 forms electric signals that may be input to one or more transducers (312 in FIG. 3) disposed in the catheter 102. In at least some embodiments, mechanical energy from the drive unit 110 may be used to drive an imaging core (306 in FIG. 3) disposed in the catheter 102. In at least some embodiments, electric signals transmitted from the one or more transducers (312 in FIG. 3) may be input to the processor 106 for processing. In at least some embodiments, the processed electric signals from the one or more transducers (312 in FIG. 3) may be displayed as one or more images on the one or more displays 112. In at least some embodiments, the processor 106 may also be used to control the functioning of one or more of the other components of the control module 104. For example, the processor 106 may be used to control at least one of the frequency or duration of the electrical signals transmitted from the pulse generator 108, the rotation rate of the imaging core (306 in FIG. 3) by the drive unit 110, the velocity or length of the pullback of the imaging core (306 in FIG. 3) by the drive unit 110, or one or more properties of one or more images formed on the one or more displays 112.

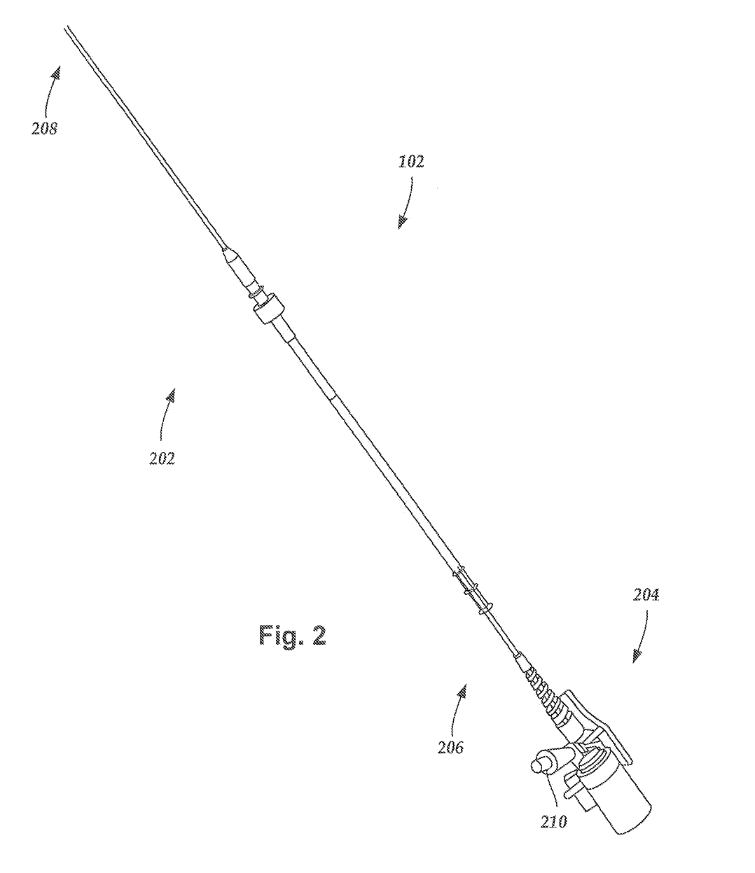

FIG. 2 is a schematic side view of one embodiment of the catheter 102 of the IVUS imaging system (100 in FIG. 1). The catheter 102 includes an elongated member 202 and a hub 204. The elongated member 202 includes a proximal end 206 and a distal end 208. In FIG. 2, the proximal end 206 of the elongated member 202 is coupled to the catheter hub 204 and the distal end 208 of the elongated member is configured and arranged for percutaneous insertion into a patient. In at least some embodiments, the catheter 102 defines at least one flush port, such as flush port 210. In at least some embodiments, the flush port 210 is defined in the hub 204. In at least some embodiments, the hub 204 is configured and arranged to couple to the control module (104 in FIG. 1). In some embodiments, the elongated member 202 and the hub 204 are formed as a unitary body. In other embodiments, the elongated member 202 and the catheter hub 204 are formed separately and subsequently assembled together.

FIG. 3 is a schematic perspective view of one embodiment of the distal end 208 of the elongated member 202 of the catheter 102. The elongated member 202 includes a sheath 302 and a lumen 304. An imaging core 306 is disposed in the lumen 304. The imaging core 306 includes an imaging device 308 coupled to a distal end of a drive cable 310.

The sheath 302 may be formed from any flexible, biocompatible material suitable for insertion into a patient. Examples of suitable materials include, for example, polyethylene, polyurethane, plastic, spiral-cut stainless steel, nitinol hypotube, and the like or combinations thereof.

One or more transducers 312 may be mounted to the imaging device 308 and employed to transmit and receive acoustic signals. In a preferred embodiment (as shown in FIG. 3), an array of transducers 312 are mounted to the imaging device 308. In other embodiments, a single transducer may be employed. In yet other embodiments, multiple transducers in an irregular-array may be employed. Any number of transducers 312 can be used. For example, there can be one, two, three, four, five, six, seven, eight, nine, ten, twelve, fifteen, sixteen, twenty, twenty-five, fifty, one hundred, five hundred, one thousand, or more transducers. As will be recognized, other numbers of transducers may also be used.

The one or more transducers 312 may be formed from one or more known materials capable of transforming applied electrical signals to pressure distortions on the surface of the one or more transducers 312, and vice versa. Examples of suitable materials include piezoelectric ceramic materials, piezocomposite materials, piezoelectric plastics, barium titanates, lead zirconate titanates, lead metaniobates, polyvinylidenefluorides, and the like.

The pressure distortions on the surface of the one or more transducers 312 form acoustic signals of a frequency based on the resonant frequencies of the one or more transducers 312. The resonant frequencies of the one or more transducers 312 may be affected by the size, shape, and material used to form the one or more transducers 312. The one or more transducers 312 may be formed in any shape suitable for positioning within the catheter 102 and for propagating acoustic signals of a desired frequency in one or more selected directions. For example, transducers may be disc-shaped, block-shaped, rectangular-shaped, oval-shaped, and the like. The one or more transducers may be formed in the desired shape by any process including, for example, dicing, dice and fill, machining, microfabrication, and the like.

As an example, each of the one or more transducers 312 may include a layer of piezoelectric material sandwiched between a conductive acoustic lens and a conductive backing material formed from an acoustically absorbent material (e.g., an epoxy substrate with tungsten particles). During operation, the piezoelectric layer may be electrically excited by both the backing material and the acoustic lens to cause the emission of acoustic signals.

In at least some embodiments, the one or more transducers 312 can be used to form a radial cross-sectional image of a surrounding space. Thus, for example, when the one or more transducers 312 are disposed in the catheter 102 and inserted into a blood vessel of a patient, the one more transducers 312 may be used to form an image of the walls of the blood vessel and tissue surrounding the blood vessel.

In at least some embodiments, the imaging core 306 may be rotated about a longitudinal axis of the catheter 102. As the imaging core 306 rotates, the one or more transducers 312 emit acoustic signals in different radial directions. When an emitted acoustic signal with sufficient energy encounters one or more medium boundaries, such as one or more tissue boundaries, a portion of the emitted acoustic signal is reflected back to the emitting transducer as an echo signal. Each echo signal that reaches a transducer with sufficient energy to be detected is transformed to an electrical signal in the receiving transducer. The one or more transformed electrical signals are transmitted to the control module (104 in FIG. 1) where the processor 106 processes the electrical-signal characteristics to form a displayable image of the imaged region based, at least in part, on a collection of information from each of the acoustic signals transmitted and the echo signals received. In at least some embodiments, the rotation of the imaging core 306 is driven by the drive unit 110 disposed in the control module (104 in FIG. 1) via the drive cable 310.

As the one or more transducers 312 rotate about the longitudinal axis of the catheter 102 emitting acoustic signals, a plurality of images are formed that collectively form a radial cross-sectional image of a portion of the region surrounding the one or more transducers 312, such as the walls of a blood vessel of interest and the tissue surrounding the blood vessel. In at least some embodiments, the radial cross-sectional image can be displayed on one or more displays 112.

In at least some embodiments, the imaging core 306 may also move longitudinally along the blood vessel within which the catheter 102 is inserted so that a plurality of cross-sectional images may be formed along an axial length of the blood vessel. In at least some embodiments, during an imaging procedure the one or more transducers 312 may be retracted (i.e., pulled back) along the longitudinal length of the catheter 102. In at least some embodiments, the catheter 102 includes at least one telescoping section that can be retracted during pullback of the one or more transducers 312. In at least some embodiments, the drive unit 110 drives the pullback of the imaging core 306 within the catheter 102. In at least some embodiments, the drive unit 110 pullback distance of the imaging core is at least 5 cm. In at least some embodiments, the drive unit 110 pullback distance of the imaging core is at least 10 cm. In at least some embodiments, the drive unit 110 pullback distance of the imaging core is at least 15 cm. In at least some embodiments, the drive unit 110 pullback distance of the imaging core is at least 20 cm. In at least some embodiments, the drive unit 110 pullback distance of the imaging core is at least 25 cm.

In at least some embodiments, one or more transducer conductors 314 electrically couple the transducers 312 to the control module 104 (See FIG. 1). In at least some embodiments, the one or more transducer conductors 314 extend along the drive cable 310.

In at least some embodiments, one or more transducers 312 may be mounted to the distal end 208 of the imaging core 308. The imaging core 308 may be inserted in the lumen of the catheter 102. In at least some embodiments, the catheter 102 (and imaging core 308) may be inserted percutaneously into a patient via an accessible blood vessel, such as the femoral artery, at a site remote from a target imaging location. The catheter 102 may then be advanced through patient vasculature to the target imaging location, such as a portion of a selected blood vessel.

Typically, the transducers 312 direct the acoustic signals, and receive echo signals, for only a relatively small region of the surrounding tissue at any given time. After receiving the backscattered echo signal from one region of a vessel or tissue, the transducers 312 are rotated (e.g., by an amount in the range of, for example, 0.5 to 2 degrees) to obtain the IVUS signal from the next region. By rotating completely around a circle in this manner, a 360° IVUS image can be generated. Each position of the transducer produces an IVUS signal which may be referred to as a “scan line.” The ongoing rotation of the transducers 312 allow the generation of “real-time” IVUS images. In at least some embodiments, the transducers 312 rotate at least one, twice, three times, five times, ten times, twenty times, or thirty times per second. Other rotation rates may also be used.

Computer-assisted methods can be employed to analyze one or more IVUS images in order to identify the component tissue types (i.e., tissue characterization). Tissue characterization may provide information beyond what may be obtainable from a visual reading of gray-level IVUS images, or “eyeballing” an IVUS image. Tissue characterization methods may enable visualization of pathologies and lesions associated with patient vasculature. Tissue characterization can also be used to monitor disease progression or patient response to therapy.

Different tissue types imprint their own “signature” on an echo signal received by the one or more transducers 312. The echo signals can be received, the signatures read, and uniquely attributed to a tissue type. Tissue characterization may involve in vitro recording of echo signal characteristics of a large number of samples of each tissue type of clinical interest. If the echo signal characteristics can be shown (by mathematical analysis) to maintain their similarity within each tissue type and distinctness between tissue types, then the echo signal characteristics can be regarded as a surrogate for tissue type. Thus, a tissue characterization system can be created by implementing an appropriate signal characterization system.

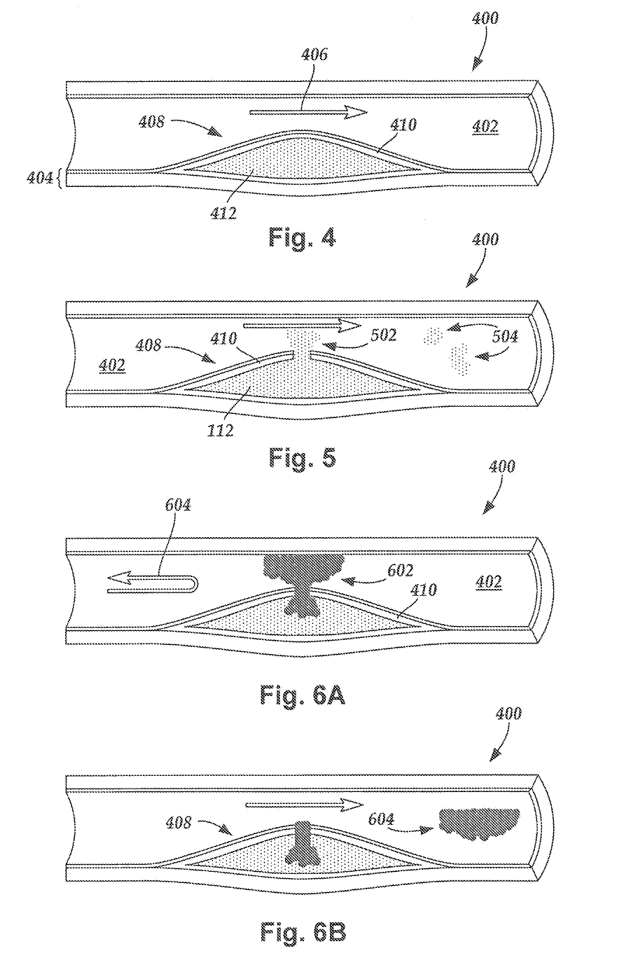

One potential clinical application of tissue characterization is the detection of vulnerable plaque (i.e., atheromata) disposed in a blood vessel. A high-risk, or vulnerable, coronary atheroma prone to rupture or erosion often includes a lipid-rich core (“core”) with an overlying thin cap infiltrated by macrophages. FIG. 4 is a schematic longitudinal cross-section of a portion of a blood vessel with an exemplary atheroma. A blood vessel 400 includes a lumen 402, a wall 404 with multiple layers of tissue, and blood flowing through the lumen 402 generally in the direction indicated by directional arrow 406. The blood vessel 400 further includes an atheroma 408 between several layers of tissue in the wall 104. The atheroma 408 includes a cap 410 and a necrotic core 412. Caps typically include one or more layers of fibrous connective tissue, and cores typically include many different types of materials, including macrophages, fatty cells, lipid-rich materials, cholesterol, calcium, foam cells, micro-calcifications, and the like.

FIG. 5 is a schematic longitudinal cross-section of the portion of the blood vessel shown in FIG. 4 having an atheroma with a ruptured cap. In FIG. 5, the cap 410 has ruptured, exposing the core 412 of the atheroma 408 to the lumen 402 of the blood vessel 400. When a cap ruptures, pieces of the core can exit the atheroma and enter the lumen of the blood vessel. For example, in FIG. 5, a portion 502 of the core 412 is extending through the ruptured cap 410 and separated pieces 504 of the core 412 are shown downstream from the atheroma 408. Separated pieces 504 of the core 412 can be transported downstream and subsequently occlude the blood vessel 400 downstream from the atheroma 408, or occlude one or more other blood vessels downstream from the blood vessel 400.

Thrombus formation may be triggered as a result of the cap rupture. FIG. 6A is a schematic longitudinal cross-section of the portion of the blood vessel shown in FIG. 4 with an occluding thrombus formed in a cap rupture. In FIG. 6A, a thrombus 602 has formed in and around the rupture of the cap 410. Sometimes a thrombus can form that is large enough to occlude a blood vessel. In FIG. 6A, the thrombus 602 has filled the rupture of the cap 410 and has expanded to occlude the lumen 402 of the blood vessel 400. In some cases, an occluding thrombus can halt the flow of blood downstream from the thrombus, as shown in FIG. 6A by U-shaped directional arrow 604. Pooling of blood may occur upstream from the atheroma which may cause many different ill-effects, such as development of an aneurism, or a tear in the wall of the blood vessel with or without subsequent internal bleeding and additional thrombus formation.

A thrombus, or a portion of a thrombus, may detach from the rupture of the cap and be transported downstream. FIG. 6B is a schematic longitudinal cross-section of the portion of the blood vessel shown in FIG. 4 with a detached thrombus. In FIG. 6B, the portion 604 of the thrombus (602 in FIG. 6A) is shown detached and transported to a location downstream from the atheroma 408. The detached portion 604 of the thrombus (602 in FIG. 6A) may subsequently occlude the blood vessel 400 downstream from the atheroma, or occlude one or more other blood vessels downstream from the blood vessel 400.

As discussed above, an atheroma with a necrotic core (“NC”) may lead to one or more adverse effects for a patient. Accordingly, when classifying tissues, the classification of a necrotic core (“NC”) may be of significant clinical interest. As mentioned above, an NC region often includes some degree of micro-calcification. The micro-calcification within an NC region may produce signal attenuation on an IVUS image. The amount of signal attenuation on an IVUS image may be proportional to the center frequency of the acoustic signals transmitted from the one or more transducers 312.

The quality of an image produced at different depths from the one or more transducers 312 may be affected by one or more factors including, for example, bandwidth, transducer focus, beam pattern, as well as the frequency of the acoustic signals. Increasing the frequency of the acoustic signals output from the one or more transducers 312 may improve the resolution of a generated image. The frequency of the acoustic signal output from the one or more transducers 312 may also affect the penetration depth of the acoustic signals output from the one or more transducers 312. In general, as the frequency of acoustic signals are lowered, the depth of the penetration of the acoustic signals within patient tissue increases.

At least some conventional IVUS imaging systems employ transducers that transmit acoustic signals having a single wideband frequency range. Employing a wideband frequency range may have some benefit of a higher resolution associated with higher frequencies, while also having some benefit of improved penetration associated with lower frequencies. Signal-to-noise ratios for certain frequencies within a wideband frequency range, however, may be inadequate for frequencies of interest, due to limitations on transducer bandwidth and peak amplitude.

An enhanced IVUS imaging technique (“imaging technique”) includes transmitting a plurality of acoustic signals, at least some of the plurality of acoustic signals having a center frequency that is different from the center frequency of at least some other of the plurality of acoustic signals. In at least some embodiments, at least some of the acoustic signals are high-frequency acoustic signals. In at least some embodiments, at least some of the acoustic signals are low-frequency acoustic signals.

In at least some embodiments, a high-frequency acoustic signal has a center frequency of at least 35 MHz, 40 MHz, 45 MHz, 50 MHz, 55 MHz, 60 MHz, 65 MHz, 70 MHz, 75 MHz, or more. In at least some embodiments, a high-frequency acoustic signal has a center frequency between 35 MHz and 55 MHz. In at least some embodiments, a high-frequency acoustic signal has a center frequency between 40 MHz and 50 MHz. In at least some embodiments, a high-frequency acoustic signal has a center frequency of 40 MHz. In at least some embodiments, a high-frequency acoustic signal has a center frequency of 50 MHz.

In at least some embodiments, a low-frequency acoustic signal has a center frequency that is no greater than 30 MHz. In at least some embodiments, a low-frequency acoustic signal has a center frequency that is no greater than 25 MHz. In at least some embodiments, a low-frequency acoustic signal has a center frequency that is no greater than 20 MHz. In at least some embodiments, a low-frequency acoustic signal has a center frequency that is no greater than 15 MHz. In at least some embodiments, a low-frequency acoustic signal has a center frequency that is no greater than 10 MHz. In at least some embodiments, a low-frequency acoustic signal has a center frequency between 10 MHz and 30 MHz. In at least some embodiments, a low-frequency acoustic signal has a center frequency between 15 MHz and 25 MHz. In at least some embodiments, a low-frequency acoustic signal has a center frequency of 25 MHz. In at least some embodiments, a low-frequency acoustic signal has a center frequency of 20 MHz.

In at least some embodiments, at least some of the plurality of acoustic signals have a center frequency that is at least 15 MHz lower than the center frequency of at least some other of the plurality of acoustic signals. In at least some embodiments, at least some of the plurality of acoustic signals have a center frequency that is at least 20 MHz lower than the center frequency of at least some other of the plurality of acoustic signals. In at least some embodiments, at least some of the plurality of acoustic signals have a center frequency that is at least 25 MHz lower than the center frequency of at least some other of the plurality of acoustic signals. In at least some embodiments, at least some of the plurality of acoustic signals have a center frequency that is at least 30 MHz lower than the center frequency of at least some other of the plurality of acoustic signals.

In at least some embodiments, the imaging technique increases the available bandwidth of received echo signals, when compared to using acoustic signals having a single wideband frequency, without producing inadequate signal-to-noise ratios (i.e., signal-to-noise ratios that prevent reliable tissue classification). In at least some embodiments, the bandwidths of the transmitted acoustic signals are configurable. In at least some embodiments, the individual fractional bandwidths of the transmitted acoustic signals are no greater than 10%, 20%, 30% of the central frequencies ranging from 20 MHz to 70 MHz. In at least some embodiments, the bandwidths of the transmitted acoustic signals overlap one another. In at least some embodiments, the acoustic-signal repetition rate may be determined by a minimal required time for a given scan depth. It also measures the signal strength at two very different frequencies (e.g., 25 MHz and 50 MHz). All bandwidths discussed herein are determined at full width at half max.

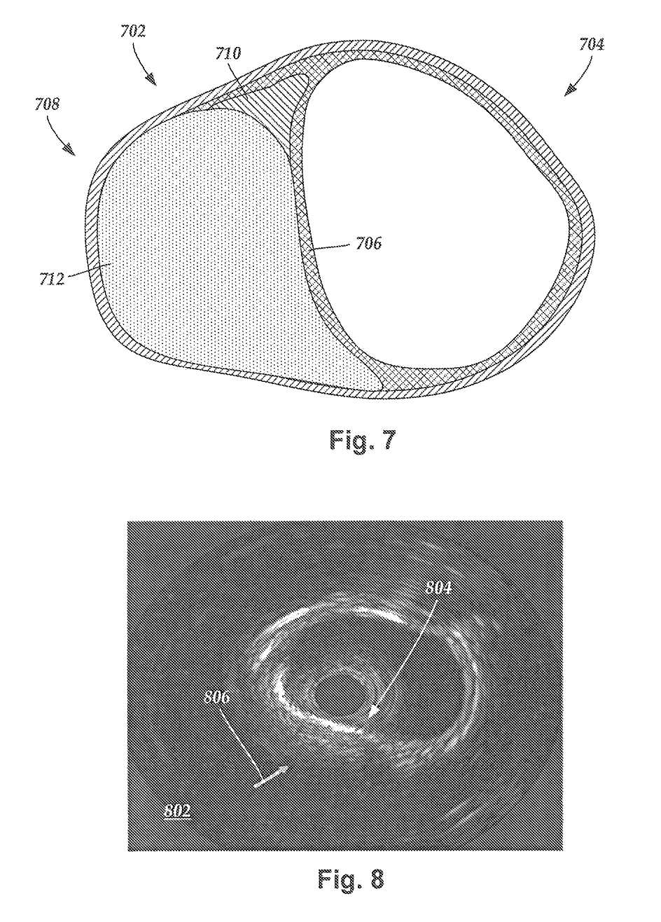

FIG. 7 is a schematic transverse cross-sectional view of another embodiment of an atheroma 702 disposed in a blood vessel 704. The atheroma 702 including a cap 706 disposed over an NC region 708, the NC region 708 including an early necrotic core 710 and a late necrotic core 712.

When an IVUS image is generated of an atheroma by transmitting high-frequency acoustic signals, the NC region may form a shadow on the IVUS image in a manner similar to a typical calcified lesion (e.g., damaged tissue). FIG. 8 shows one embodiment of an IVUS image 802 that includes an atheroma 804 with an NC region 806 (shown in FIG. 8 by an arrow). The IVUS image 802 is generated by transmitting high-frequency acoustic signals. The atheroma 804 has an appearance that resembles a typical calcified lesion, with a layer of visible echoes and a shadow behind the layer of visibly echoes corresponding to the NC region 806. The degree of attenuation caused by the NC region 806 may depend on one or more factors including, for example, the amount of micro-calcification within the NC region 806, the thickness of NC region 806, the angle of incident of the acoustic signals, or the like. As shown in FIG. 8, when the IVUS image 802 is generated using high-frequency acoustic signals, the NC region 806 may include a significant amount of attenuation that may hinder the efficacy of tissue classification.

Imaging an atheroma using low-frequency acoustic signals may reduce shadowing within NC regions. When an IVUS image is generated of an atheroma by transmitting low-frequency acoustic signals, the acoustic signals can often penetrate the NC region without creating an acoustic shadow. Accordingly, using low-frequency acoustic signals may improve tissue classification when imaging an atheroma with a high degree of attenuation. IVUS images generated using low-frequency acoustic signals, however, may have decreased resolution, as compared to IVUS images generated using high-frequency acoustic signals.

In some embodiments, the imaging technique includes, for each scan line, transmitting at least one acoustic signal having a first center frequency and at least one acoustic signal having a second center frequency that is different from the first frequency for each scan line during an imaging procedure. It will be understood that the relative number of each frequency of acoustic signals may vary.

In other embodiments, the imaging technique transmits acoustic signals with a first center frequency along a first scan line and acoustic signals with a second center frequency along a second scan line. When each scan line includes only acoustic signals having one given center frequency, the acoustic signals may be transmitted using a repeating pattern between a series of scan lines. Any transmission pattern may be employed including, for example, a) transmitting only one or more high-frequency signals along odd scan lines and transmitting only one or more low-frequency signals along even scan lines, b) transmitting only one or more high-frequency signals along even scan lines and transmitting only one or more low-frequency signals along odd scan lines, c) transmitting only one or more high-frequency signals along two or more adjacent scan lines and transmitting only one or more low-frequency signals along two or more other adjacent scan lines, d) transmitting only one or more high-frequency signals along every Nth scan line (where N is a whole number greater than 2), e) transmitting only one or more low-frequency signals along every Nth scan line (where N is a whole number greater than 2), f) transmitting only one or more high-frequency signals along a given sector of a scanning revolution and transmitting only one or more low-frequency signals along another sector of the scanning revolution, or the like.

Any number of acoustic signals may be transmitted from the transducers 312. The transmitted acoustic signals may include any number of different center frequencies. The transducers 312 may be configured and arranged for transmitting acoustic signals having two, three, four, five, six, or more different center frequencies. It will be understood that the transducers 312 may be configured and arranged for transmitting acoustic signals that include more than six center frequencies.

It may be an advantage to transmit at least one high-frequency acoustic signal and at least one low-frequency signal during an imaging procedure. The high-frequency acoustic signal may be particularly useful to improve resolution of the image as compared to the low-frequency signal, and the low-frequency may be useful to image an NC region behind a cap, which may be shrouded in a shadow when a high-frequency acoustic signal is used alone. Additionally, by transmitting multiple acoustic signals, each at different frequency ranges, the detrimental signal-to-noise ratios obtained using a single wideband frequency may be avoided.

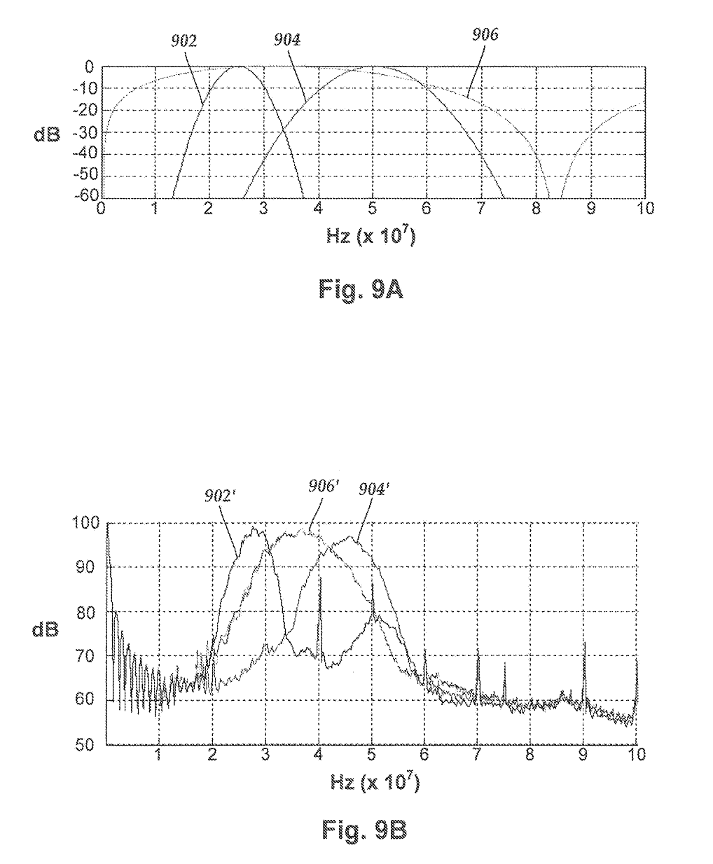

FIG. 9A is a graph showing spectra of acoustic signals having different center frequencies, the acoustic signals suitable for transmission from one or more transducers during an imaging procedure. In FIG. 9A, a first acoustic signal 902 is a low-frequency signal having a center frequency of 25 MHz and a bandwidth of approximately 7.5 MHz. A second acoustic signal 904 is a high-frequency signal having a center frequency of 50 MHz and a bandwidth of approximately 15 MHz. As a comparison, a wideband signal 906 with a center frequency of approximately 40 MHz is shown in FIG. 9A with a bandwidth of approximately 45 MHz

FIG. 9B is a graph showing spectra of exemplary echo signals received by one or more transducers after reflection of the acoustic signals 902, 904, and 906 from patient tissue. Echo signal 902′ corresponds to acoustic signal 902; echo signal 904′ corresponds to acoustic signal 904; and echo signal 906′ corresponds to wideband signal 906. FIG. 9B shows that the relative strength of the echo signal 902′ is approximately 10 dB higher than the echo signal 906′ at 25 MHz. FIG. 9B also shows that the relative strength of the echo signal 904′ is approximately 10 dB higher than the echo signal 906′ at 50 MHz.

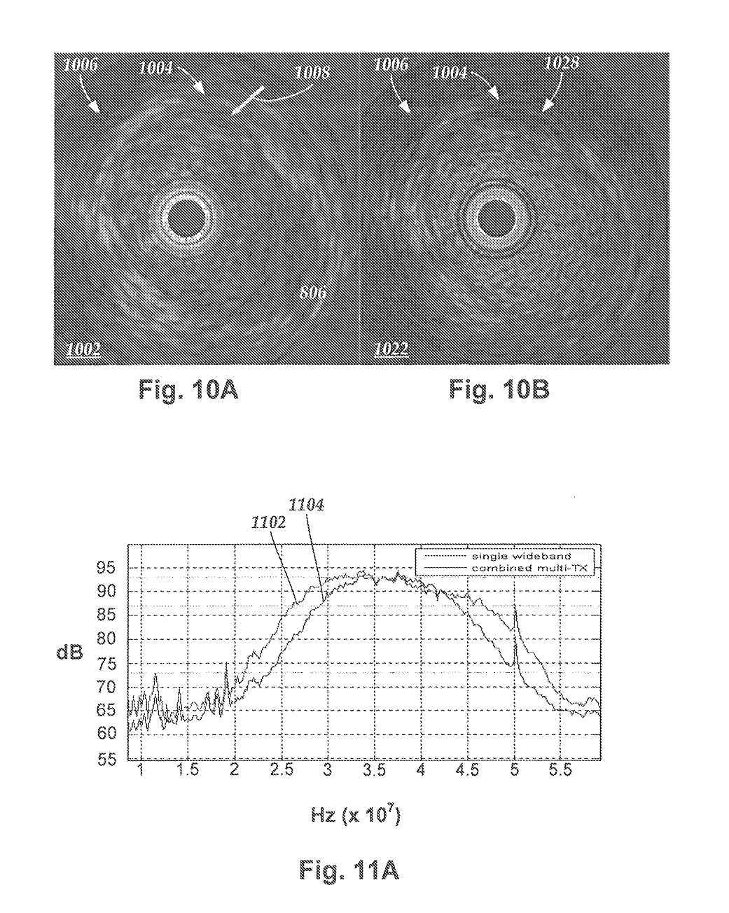

FIGS. 10A and 10B provide an example of different appearances of an atheroma obtained using different frequencies of acoustic signals. FIGS. 10A and 10B are schematic views of IVUS images showing an atheroma 1004 within a blood vessel 1006. FIG. 10A shows one embodiment of an IVUS image 1002 generated using acoustic signals transmitted at a first center frequency. The first center frequency is a low-frequency acoustic signal (e.g., having a center frequency of 25 MHz). FIG. 10B shows one embodiment of an IVUS image 1022 generated using acoustic signals transmitted at a second center frequency that is greater than the first center frequency. In FIG. 10B, the second center frequency is a high-frequency acoustic signal.

A comparison of FIG. 10A and FIG. 10B demonstrates that, because of the frequency dependencies of ultrasound scattering, or attenuation, or both, imaging an atheroma at both low and high frequencies may provide useful information for enhancing tissue characterization. Although the resolution of FIG. 10B is greater than the resolution of FIG. 10A, a comparison of FIG. 10A to FIG. 10B, however, reveals that potentially useful information for tissue classification is visible in FIG. 10A, but not in FIG. 10B. In FIG. 10A, an adventitia wall 1008 (shown in FIG. 10A by an arrow) is visible. In FIG. 10B, however, a shadow (1028 in FIG. 10B) obscures the adventitia wall (1008 in FIG. 10A).

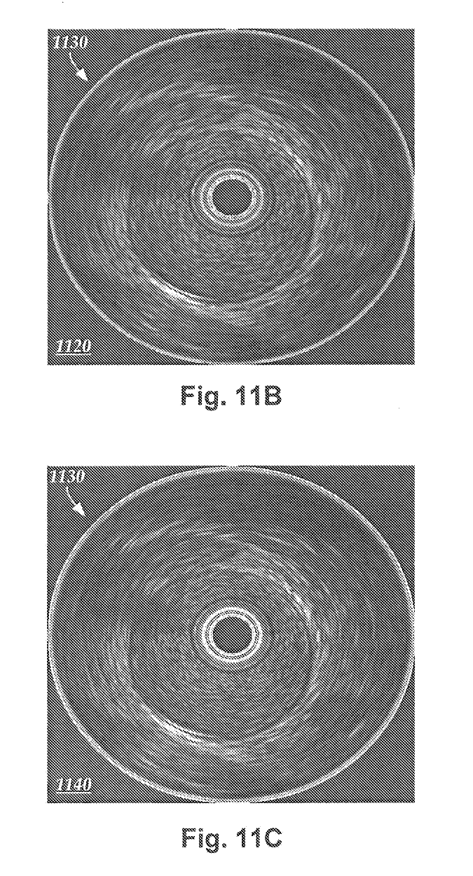

FIGS. 11A-11C illustrate an example of potential differences between an IVUS image of a blood vessel generated from echo signals received in response to the transmission of acoustic signals having a single wideband frequency and an IVUS image of the same blood vessel generated from a combination of the echo signals received in response to the transmission of acoustic signals at multiple different frequencies. FIG. 11A is a graph showing exemplary spectra of echo signals received by one or more transducers after reflection of acoustic signals from patient tissue. An acoustic signal 1102 is a combined signal from a low-frequency signal and a high-frequency signal. In FIG. 11A, the low-frequency signal has a center frequency of 25 MHz and a 30% bandwidth, and the high-frequency signal has a center frequency of 50 MHz and a 30% bandwidth. For comparison, a wideband signal 1104 is shown in FIG. 11A having a center frequency of 40 MHz and a full bandwidth. FIG. 11B shows one embodiment of an IVUS image 1120 of a blood vessel 1130 generated using the single wideband signal 1104. FIG. 11C shows one embodiment of an IVUS image 1140 of the blood vessel 1130 generated using the combined acoustic signals 1102. A comparison of the IVUS image 1120 to the IVUS image 1140 reveals a finer texture in the IVUS image 1140 than the IVUS image 1120 along an axial direction of the blood vessel 1130.

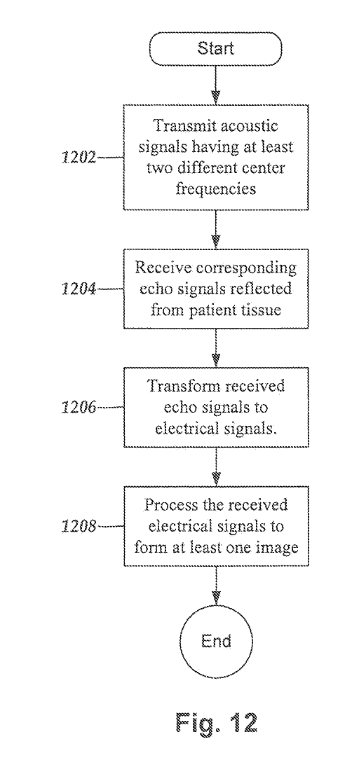

FIG. 12 is a flow diagram showing one exemplary embodiment of an enhanced IVUS imaging technique. In step 1202, acoustic signals having at least two different center frequencies are transmitted along a series of scan lines towards patient tissue between incremental rotations of the transducer. In at least some embodiments, at least one of the acoustic signals has a frequency bandwidth centered at a first frequency and at least one of the acoustic signals has a frequency bandwidth centered at a second frequency that is lower than the first frequency. In at least some embodiments, the first frequency is a high frequency and the second frequency is a low frequency. In step 1204, for each scan line, corresponding echo signals reflected from patient tissue are received by the transducer. In step 1206, the received echo signals are transformed to electrical signals. In step 1208, the received electrical signals are processed from the transducer to form at least one image.

It will be understood that each block of the flowchart illustrations, and combinations of blocks in the flowchart illustrations, as well any portion of the tissue classifier, imager, control module, systems and methods disclosed herein, can be implemented by computer program instructions. These program instructions may be provided to a processor to produce a machine, such that the instructions, which execute on the processor, create means for implementing the actions specified in the flowchart block or blocks or described for the tissue classifier, imager, control module, systems and methods disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

In alternate embodiments, the imaging technique may be implemented in different ways. In at least some embodiments, the imaging technique may be employed to improve the multiple frequency method for blood suppression, especially for IVUS transducers having limited bandwidth. In at least some embodiments, the imaging technique can be combined with one or more other techniques, such as coded excitation to maximize the signal-to-noise ratio. In at least some embodiments, the imaging technique can be used to improve identification or classification of one or more structures located behind calcium deposits. In at least some embodiments, the imaging technique can be employed to improve identification behind other objects, such as one or more structures positioned behind a guidewire. In at least some embodiments, the imaging technique may be used to improve quantification of tissue attenuation due to the significant improvement on signal-to-noise ratio. In at least some embodiments, the imaging technique may be used to improve border detection in a structure (e.g., an atheroma, a blood vessel, or the like). In at least some embodiments, the imaging technique may be employed to improve ultrasound elastography by providing better granularity to select the appropriate time step size for estimating the induced strain from the cardiac cycle.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A method for imaging patient tissue using an intravascular ultrasound image, the method comprising:
inserting a catheter into a patient blood vessel, the catheter comprising an imaging core configured and arranged for insertion into a lumen of the catheter and disposition at a distal end of the catheter, the imaging core comprising at least one ultrasound transducer configured and arranged for transforming applied electrical signals to a plurality of acoustic signals;
transmitting the acoustic signals along a series of scan lines towards patient tissue between incremental rotations of the at least one transducer, wherein a plurality of the acoustic signals transmitted along the series of scan lines are first acoustic signals having first frequency bandwidths centered at a first center frequency, and wherein a plurality of the acoustic signals transmitted along the series of scan lines are second acoustic signals having second frequency bandwidths centered at a second center frequency that is lower than the first center frequency;
for each scan line, receiving corresponding echo signals reflected from patient tissue;
transforming the received echo signals to electrical signals;
processing the received electrical signals from the at least one transducer to form at least one image; and
displaying the at least one image on a display.

2. The method of claim 1, wherein transmitting the acoustic signals along a series of scan lines towards patient tissue between incremental rotations of the at least one transducer comprises, for each scan line, transmitting at least one of the first acoustic signals and at least one of the second acoustic signals.

3. The method of claim 1, wherein sequentially transmitting the acoustic signals along a series of scan lines towards patient tissue between incremental rotations of the at least one transducer comprises, for each scan line, transmitting at least one of the first acoustic signals or at least one of the second acoustic signals.

4. The method of claim 1, wherein sequentially transmitting the acoustic signals along a series of scan lines towards patient tissue between incremental rotations of the at least one transducer comprises, for each two adjacent scan lines, transmitting at least one of the first acoustic signals along one of the two adjacent scan lines and transmitting at least one of the second acoustic signals along the other of the two adjacent scan lines.

5. The method of claim 1, wherein sequentially transmitting the acoustic signals along a series of scan lines towards patient tissue between incremental rotations of the at least one transducer comprises, for each first pair of adjacent scan lines, transmitting at least one of the first acoustic signals, and for each second pair of adjacent scan lines positioned adjacent the first pair of adjacent scan lines, transmitting at least one of the second acoustic signals.

6. The method of claim 1, wherein sequentially transmitting the acoustic signals along a series of scan lines towards patient tissue between incremental rotations of the at least one transducer comprises transmitting at least one first acoustic signal along every Nth scan line (where N is a whole number greater than 2) and transmitting at least one second acoustic signal along each of the remaining scan lines.

7. The method of claim 1, wherein sequentially transmitting the acoustic signals along a series of scan lines towards patient tissue between incremental rotations of the at least one transducer comprises transmitting at least one second acoustic signal along every Nth scan line (where N is a whole number greater than 2) and transmitting at least one first acoustic signal along each of the remaining scan lines.

8. The method of claim 1, wherein sequentially transmitting the acoustic signals along a series of scan lines towards patient tissue between incremental rotations of the at least one transducer comprises transmitting at least one of the first acoustic signals along a first sector of a scanning revolution and transmitting at least one of the second acoustic signals along another sector of the scanning revolution.

9. The method of claim 1, wherein the second center frequency is at least 20 MHz lower than the first center frequency.

10. The method of claim 1, wherein the second frequency is selected such that, when a necrotic region of an atheroma is imaged, the second acoustic signals penetrate the necrotic region of the atheroma to image of the necrotic region.

11. The method of claim 1, wherein at least one of the first frequency bandwidths or the second frequency bandwidths are configurable.

12. The method of claim 1, wherein the first frequency bandwidths overlap with the second frequency bandwidths.

13. A computer-readable medium having processor-executable instructions for generating an intravascular ultrasound image formed in response to transmission of a plurality of acoustic signals from a transducer, the processor-executable instructions when installed onto a device enable the device to perform actions, comprising:
transmitting the acoustic signals along a series of scan lines towards patient tissue between incremental rotations of the at least one transducer, wherein at least some of the acoustic signals transmitted along the series of scan lines are first frequency acoustic signals having first frequency bandwidths centered at a first center frequency, and wherein at least some of the acoustic signals transmitted along the series of scan lines are second frequency acoustic signals having second frequency bandwidths centered at a second center frequency that is lower than the first center frequency;
for each scan line, receiving corresponding echo signals reflected from patient tissue;
transforming the received echo signals to electrical signals;
processing the received electrical signals from the transducer to form at least one image; and
displaying the at least one image on a display.

14. The computer-readable medium of claim 13, wherein the processor-executable instructions when installed onto the device further enable, for each scan line, transmitting at least one of the first acoustic signals and at least one of the second acoustic signals.

15. The computer-readable medium of claim 13, wherein the processor-executable instructions when installed onto the device further enable, for each scan line, transmitting at least one of the first acoustic signals or at least one of the second acoustic signals.

16. The computer-readable medium of claim 13, wherein the processor-executable instructions when installed onto the device further enable, for each scan line, transmitting at least one of the first acoustic signals along one of the two adjacent scan lines and transmitting at least one of the second acoustic signals along the other of the two adjacent scan lines.

17. A catheter-based intravascular ultrasound imaging system comprising:
at least one imager disposed in a catheter at least partially insertable into a patient blood vessel, the at least one imager coupled to a control module; and
a processor in communication with the control module, the processor for executing processor-readable instructions that enable actions, including:
transmitting acoustic signals along a series of scan lines towards patient tissue between incremental rotations of the at least one transducer, wherein at least some of the acoustic signals transmitted along the series of scan lines are first frequency acoustic signals having first frequency bandwidths centered at a first center frequency, and wherein at least some of the acoustic signals transmitted along the series of scan lines are second frequency acoustic signals having second frequency bandwidths centered at a second center frequency that is lower than the first center frequency;
for each scan line, receiving corresponding echo signals reflected from patient tissue;
transforming the received echo signals to electrical signals; and
processing the received electrical signals from the imager to form at least one image; and
displaying the at least one image on a coupled display.

18. The catheter-based intravascular ultrasound imaging system of claim 17, wherein the processor for executing processor-readable instructions further enables, for each scan line, transmitting at least one of the first acoustic signals and at least one of the second acoustic signals.

19. The catheter-based intravascular ultrasound imaging system of claim 17, wherein the processor for executing processor-readable instructions further enables, for each scan line, transmitting at least one of the first acoustic signals or at least one of the second acoustic signals.

20. The catheter-based intravascular ultrasound imaging system of claim 17, wherein the processor for executing processor-readable instructions further enables, for each scan line, transmitting at least one of the first acoustic signals along one of the two adjacent scan lines and transmitting at least one of the second acoustic signals along the other of the two adjacent scan lines.
